(19) United States
(12) Patent Application Publication
(10) Pub. No.: US 2024/0038357 A1
(43) Pub. Date: Feb. 1, 2024

(54) SYSTEMS AND METHODS FOR MEDICATION ADHERENCE

(71) Applicant: LATTICE HEALTH SYSTEMS, INC., AUSTIN, TX (US)

(72) Inventors: Dale TONOGAI, LOS ALTOS, CA (US); Pramod MADABHUSHI, AUSTIN, TX (US)

(21) Appl. No.: 18/484,083

(22) Filed: Oct. 10, 2023

Related U.S. Application Data
(63) Continuation of application No. 16/657,457, filed on Oct. 18, 2019.
Application No. 16/657,457 is a continuation of application No. 15/340,652, filed on Nov. 1, 2016, now abandoned.
(60) Provisional application No. 62/249,808, filed on Nov. 2, 2015.

(51) Int. Cl. G16H 20/13; G06T 7/00; G06T 1/00

(52) U.S. Cl. CPC G16H 20/13; G06T 7/0012; G06T 1/0007; G06T 2207/30004

(57) ABSTRACT

A medication adherence service receives patients' medication schedules from health care professionals and systems. A medication adherence device is communicatively coupled to the medication adherence service. Based upon a patient's prescribed medication schedule, the medication adherence device manages and monitors a patient's adherence to the prescribed medication schedule. The medication adherence device captures images of compartments holding medications in accordance with the prescribed medication schedule. These images are analyzed to aid the patient in filling the compartments and to manage and monitor the patient in adhering to the prescribed schedule.

### CROSS-REFERENCE TO RELATED APPLICATIONS

This application is a continuation of U.S. patent application Ser. No. 16/657,457, filed Oct. 18, 2019 and entitled “SYSTEMS AND METHODS FOR MEDICATION ADHERENCE”, which is a continuation of U.S. patent application Ser. No. 15/340,652, filed Nov. 1, 2016 and entitled “SYSTEMS AND METHODS FOR MEDICATION ADHERENCE”, which claims priority from U.S. Provisional Patent Application No. 62/249,808, filed on Nov. 2, 2015 and entitled “SYSTEMS AND METHODS FOR MEDICATION ADHERENCE”, each of which is incorporated herein by reference in its entirety.

### TECHNICAL FIELD

Systems and methods for managing medication adherence are disclosed.

### BACKGROUND

A high percentage of the population is prescribed medications by their physicians. Prescriptions are often for chronic conditions, such that patients are directed to take medications on at least a daily basis, if not several times each day. Many patients are prescribed several medications for one or more conditions. To be most effective and to avoid adverse effects it is important that medications be taken on the schedule that the physician prescribes.

Medication organizers, sometimes called “pill boxes”, exist to help patients organize their medications and adhere to a schedule. While somewhat helpful, existing medication organizers lack capabilities to ensure that the organizer is properly filled with medications and that the patient is taking medication as scheduled. Moreover, existing medication organizers cannot be remotely monitored.

### SUMMARY

Embodiments described herein provide systems and methods for managing and monitoring patient adherence to medication schedules.

In one example embodiment, a medication adherence device is communicatively coupled to a medication adherence service to receive a patient medication schedule and to send patient medication adherence data to the medication adherence service. The medication adherence device has a plurality of medication compartments for organizing a patient's medications according to a prescribed medication schedule. The medication adherence device manages and monitors the patient's adherence to the prescribed medication schedule. The device has at least one interface for outputting information to the patient.

One method provides for filling a medication adherence device with medications. The medication adherence device prompts to fill an element with a desired set of contents. One or more of the medication adherence device's cameras are used to capture images of the element. The captured images are analyzed and compared against the desired contents to determine if the element has been properly filled. If the analysis concludes that the element has been properly it is recorded as such.

An example method is also provided for managing and monitoring a patient's adherence to a prescribed medication schedule using a medication adherence device. The medication adherence device prompts the patient to take a medication according to a schedule the medication adherence device has received from a medication adherence service. One or more of the medication adherence device's cameras capture an image of the medication adherence device's medication compartments. The captured images are then analyzed to determine whether the medication has been properly taken. If the analysis concludes that the medication has been properly taken it is recorded as such.

### BRIEF DESCRIPTION OF THE DRAWINGS

FIG. 1 shows a top view of an example of a prior art medication organizer.

FIG. 2 shows a top view of a second example of a prior art medication organizer.

FIG. 3 is a simplified side schematic of a first example medication adherence device, illustrating some components.

FIG. 4 is a simplified side schematic of a second example medication adherence device, illustrating some components.

FIG. 5 is a simplified front schematic of the first example medication adherence device, illustrating some components.

FIG. 6 is a simplified side schematic of a third example medication adherence device, illustrating some components.

FIG. 7 is a simplified top schematic of the third example medication adherence device, illustrating components.

FIG. 8 is another simplified top schematic of the third example medication adherence device, illustrating some components.

FIG. 9 is a simplified diagram illustrating a medication adherence device interfacing with a medication adherence service.

FIG. 10 is a flowchart illustrating an exemplary method for provisioning a medication adherence system with patient medication adherence data in accordance with an embodiment of the invention.

FIG. 11 is a flowchart illustrating an exemplary method for filling a medication adherence device in accordance with an embodiment of the invention.

FIG. 12 is a flowchart illustrating an exemplary method for alerting and monitoring a patient regarding medication adherence in accordance with an embodiment of the invention.

### DETAILED DESCRIPTION

This disclosure relates to a device to aid patients, on an ongoing basis, in properly taking medications. The device also enables management and remote monitoring of the patient's adherence to their medication schedule.

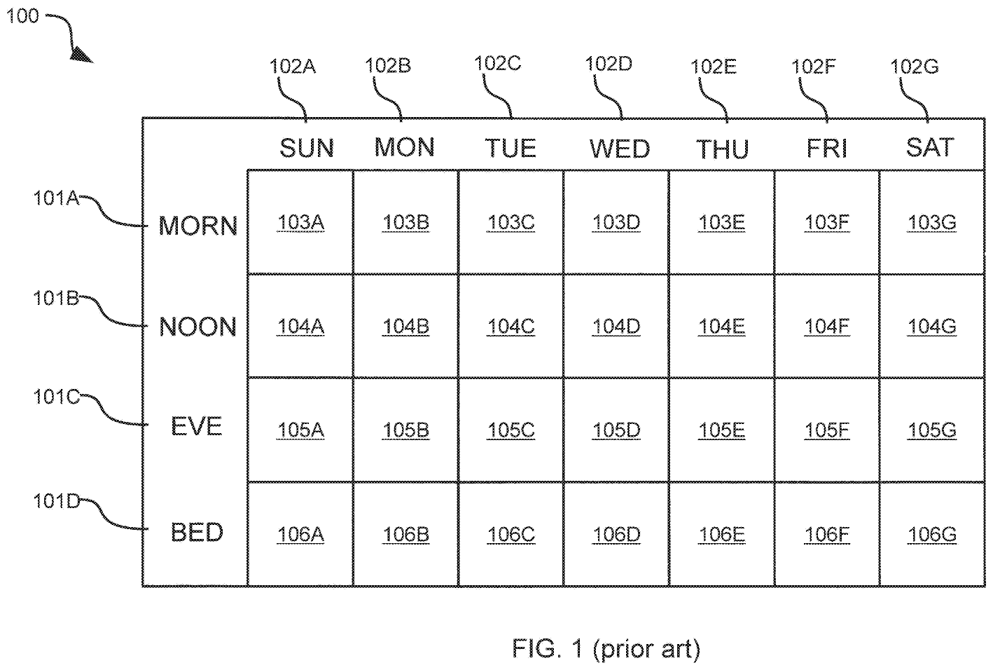

For background, FIG. 1 illustrates a top view of a prior art medication organizer 100. The organizer comprises compartments for storing medications according to the day and time they are to be taken. The example organizer is designed for a patient who takes medications four times a day. There are four rows, which are labeled according to the time that the medications placed in the compartment are to be taken: “MORN” 101A for morning, “NOON” 101B for noon, “EVE” 101C for evening and “BED” 101D for bedtime. There is a column for each day of the week, labeled “SUN” 102A for Sunday thru “SAT” 102G for Saturday. Once a week the patient fills this organizer with medications, according to the day and time the prescription indicates they are to be taken. For example, if a medication is to be taken in the morning each day of the week, the patient is expected to place the prescribed amount of the medication in each of the seven morning compartments 103A thru 103G. This process is to be completed for all the patients' medications, with a goal of having each of the medications the patient is to take that week in the correct compartment, so as to make it easy for the patient to adhere to the correct schedule for taking their medications. Unfortunately, the prior art medication organizer is not capable, beyond the compartment labels, of aiding the patient in correctly filling the medication organizer.

When it is time for the patient to take a medication, the patient selects the correct compartment to take medication from, based upon the current day and time. For example, if it is Sunday morning, they remove medications from compartment 103A which is in the compartment at the intersection of the “MORN” 101A row and the “SUN” 102A column. This same process is repeated at other times of day. Other than the labels, the compartments being empty or full, and the patient's awareness of the current day and time, there is nothing to aid the patient in adhering to the schedule for taking the medications. Moreover, the prior art has no capability to verify that it was used properly.

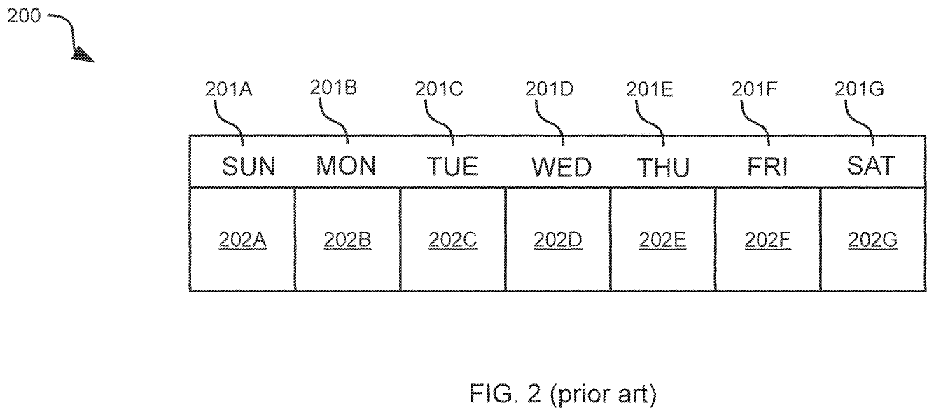

FIG. 2 is another example of a prior art medication organizer. Medication organizer 200 is designed for a patient taking medications once a day so it has a single row of compartments. There are compartments 202A thru 202G, one for each day of the week which are identified by each of the labels 201A thru 201G above the compartment. This prior art medication organizer works in a similar fashion to the previously described medication organizer 100. Medication organizers may be configured to have other rows and columns, based upon the patient's medication schedule.

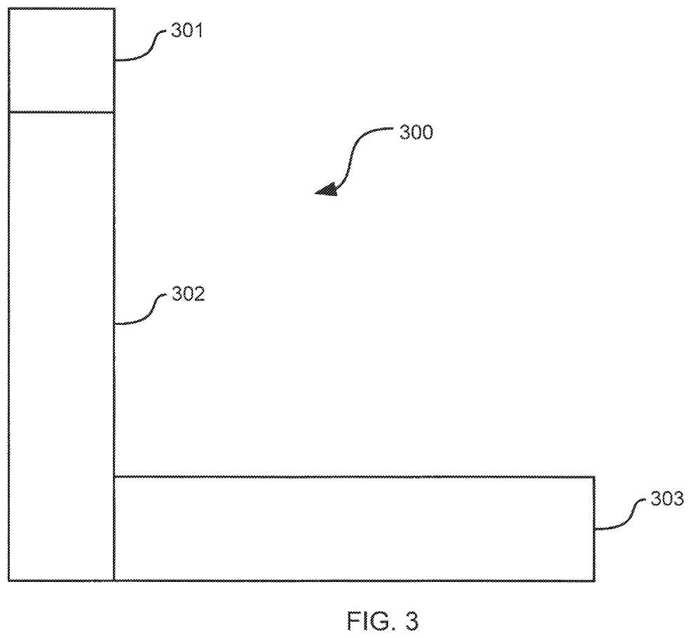

Turning now to the invention described in this disclosure, FIG. 3 is a simplified side schematic of an example medication adherence device 300, showing three high level components it is comprised of: a camera unit 301, interface unit 302, and medication unit 303.

Medication unit 303 is comprised of compartments to hold a patient's medications according to the schedule prescribed for taking the medications. The compartments may be organized in a matrix of rows and columns, with rows representing times to take medications and columns representing days of the week, similar to medication organizer 100. Alternatively, the compartments may be organized in any fashion which conveys the prescription schedule, including, for example, circular or stackable configurations. In some examples, medication unit 303 is comprised of electronic components, such as LEDs. In other examples, medication unit 303 is mechanical without any electronic components. Medication unit 303 may be attached to other components, as illustrated in the example device 300 by its attachment to interface unit 302. Alternatively, medication unit 303 may not attach to example device 300. Instead, it may be positioned to operate with the other components, as will subsequently be described. In yet another example, medication unit 303 may be docked or undocked depending upon the needs of the patient; that is, attached or unattached to other components of medication adherence device 300.

Interface unit 302 is comprised of elements to convey information to the patient or to receive information. These include one or more interfaces such as a microphone, speaker, display, touchscreen, LEDs, and the like.

Camera unit 301 comprises one or more digital cameras that are used to monitor medication unit 303 and its use by the patient. In some examples, camera unit 301 may also comprise one or more lights to illuminate medication unit 303. This is beneficial for image quality, and it may also be helpful to the patient using the medication unit to clearly view it. Lights may also be used to illuminate specific compartments of medication unit 303, as a way to direct the patient's attention and provide them with guidance. Cameras in camera unit 301 are pointed at an angle to view and capture images of the medication compartments comprising medication unit 303 and the compartments' contents. Where necessary, material comprising medication unit 303 is transparent so medications inside the compartments are viewable even when a compartment's lid is closed. Some embodiments of medication unit 303 may have compartments that are always opened, without lids. This not only provides camera unit 301 with a clear view, but lids may be unnecessary if the medication unit 303 is generally stationary, such there is minimal concern with the patient spilling the unit's contents. A medication unit 303 without compartment lids may also be easier for patients with limited dexterity to operate. In examples where the camera angle does not provide a complete view of all the compartments, some medication units 303 may have compartments with reflective walls, which would otherwise have hidden areas, such that reflected images of hidden areas in the compartment will be viewable by cameras in camera unit 301.

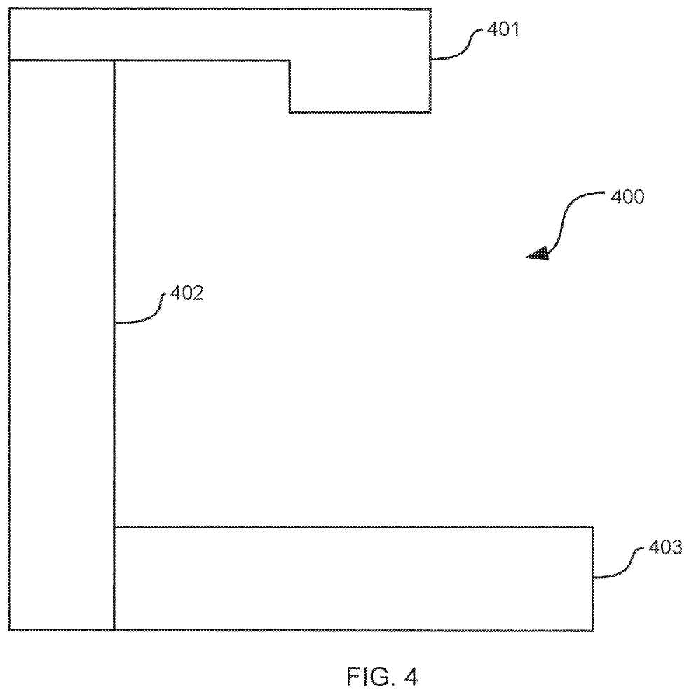

FIG. 4 is a simplified side schematic of another example medication adherence device 400, comprising medication unit 403, interface unit 402, and camera unit 401. These units are similar to their counterparts illustrated for medication adherence device 300 in FIG. 3. The principal difference with this example is the mounting and positioning of camera unit 401. In this example, the camera unit is positioned above medication unit 403, providing it a clear view of the compartments. These are only two examples of how the camera unit may be mounted and positioned—there are many other possible configurations.

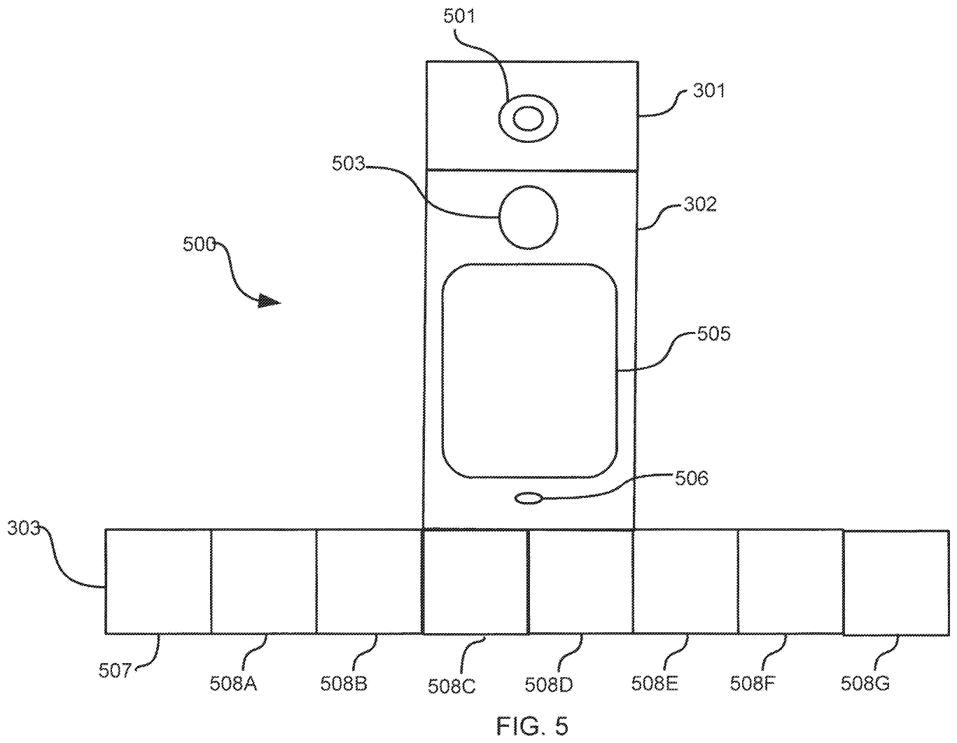

FIG. 5 is a simplified schematic of the front of medication adherence device 300, first illustrated in FIG. 3. Medication unit 303, interface unit 302, and camera unit 301 are shown as viewed from the front, with some elements each is comprised of also illustrated.

In FIG. 5 the division of medication unit 303 into compartments is shown from the front. There are seven medication compartments 508A thru 508G as viewed from the front. 507 is space to the left of the medication compartments which may be used for other purposes. The top surface of 507 may be used to label each of the rows of medication compartments that are to the right, starting with 508A, in medication unit 303. Individual medication compartments 508A thru 508G, and any other medication compartments comprising medication until 303, may be colored to covey information. For example, a compartment's color may indicate the time and/or day that medications placed into the compartment are to be taken.

The front view of interface unit 302, illustrated in FIG. 5, shows example elements comprising the interface unit. Speaker 503 is used by the medication adherence device to provide information to the patient in audio form. Display 505 is used to provide information to the patient in text or video form. In some embodiments display 505 has touchscreen capabilities, allowing the patient to respond to prompts or enter information. In some embodiments interface unit 302 is also comprised of a camera (not shown), which is in addition to camera unit 301. This additional camera may be directed towards the patient and may be used to remotely assist or monitor the patient using the device. For example, this additional camera or camera unit 301 may be used to read the prescription bottle that the patient is holding, or has otherwise placed in front of the camera, so that instructions can then be given to the patient for that specific prescription bottle. In another example, the camera allows a remote party helping the patient, such as a nurse or adult child, to view the patient's expressions and observe what they are doing at the medication adherence device. Microphone 506 is the final example element illustrated on FIG. 5 and gives interface unit 302 the capability to receive voice commands or other audio information from the patient.

FIG. 5's front view of camera unit 301 illustrates camera 501. As explained earlier, the camera is directed at an angle to view the medication compartments in medication unit 303.

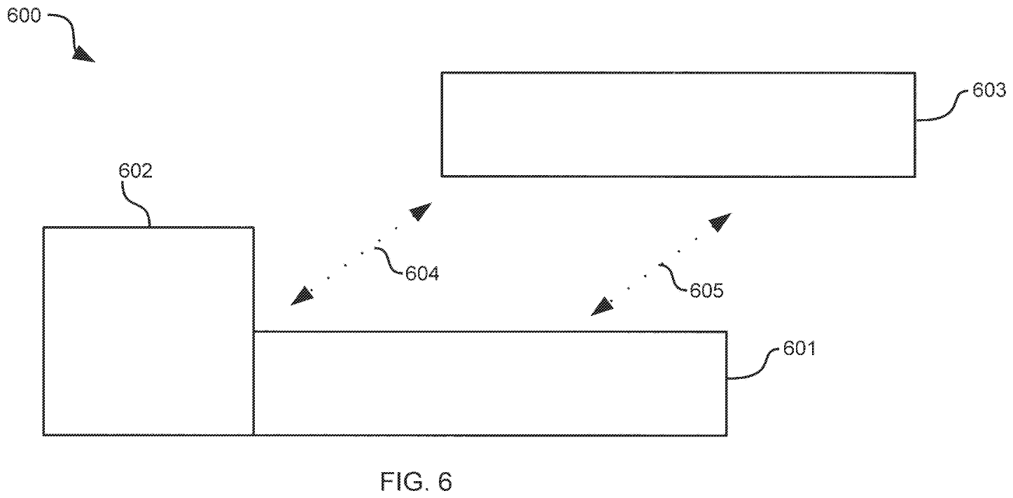

A third example of a medication adherence device is illustrated by the simplified side schematic of FIG. 6. Medication adherence device 600 is comprised of camera unit 601, interface unit 602, and medication unit 603, which provide functions similar to their counterparts in the earlier examples of medication adherence devices. However, in this example, the placement of the three units is different than the earlier examples. Camera unit 601 is positioned in front of interface unit 602 and below medication unit 603. The camera unit 601 is directed to view medication unit 603 from the bottom, with the bottom of the medication unit's medication compartments being transparent so their contents can be viewed from the bottom. In this illustration, medication unit 603 is undocked from the other components of the device. Arrows 604 and 605 indicate that the medication unit 603 can be docked or undocked from the other components. Medication unit 603 may have wireless capabilities, such as BLE, allowing the medication unit 603 to communicate even when it is not docked. For example, interface unit 602 may have a wireless interface to control LEDs on medication unit 603. One or more LEDs may be turned on, off, or flashed when reminding or directing the patient to fill the medication unit or to take medication.

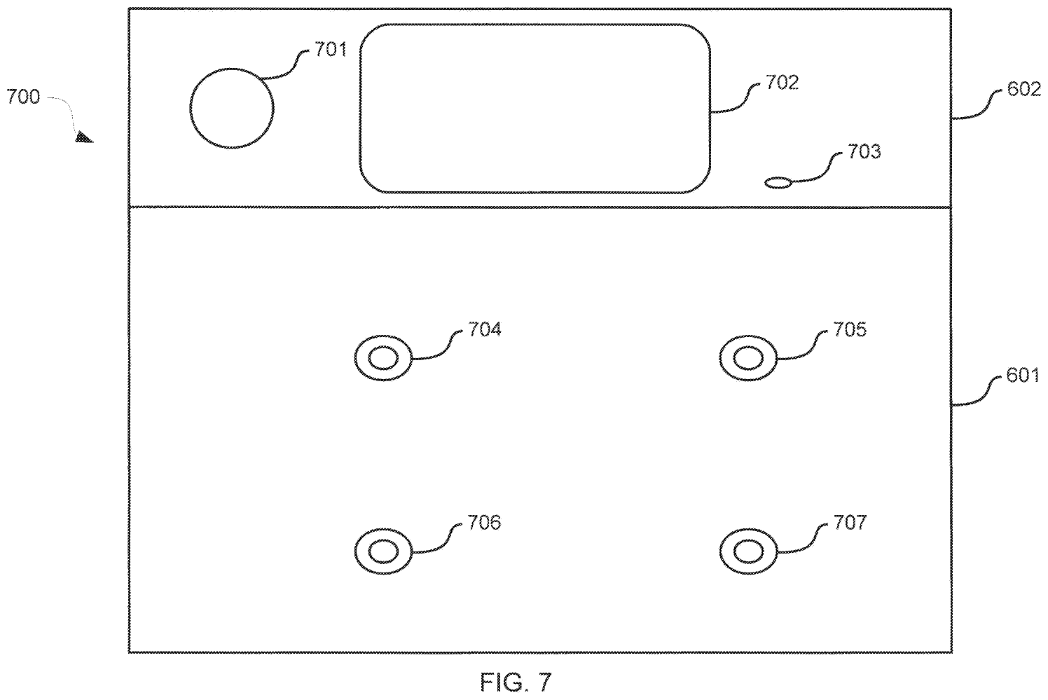

FIG. 7 is a simplified schematic of this third example of a medication adherence device, showing camera unit 601 and interface unit 602 from the top. In this illustration the Medication unit 603 is not show in FIG. 7, in order to illustrate the camera unit with the medication unit undocked. In this example, camera unit 601 is comprised of four cameras. Fewer or more cameras may be required to capture images of medication unit 603's medication compartments. The number of cameras required will depend upon the viewing angle of the camera lens and the distance between the camera and the bottom of medication unit 603. Interface unit 602 in this example is shown to have speaker 701, display 702, and microphone 703. These elements have similar functions as in the earlier examples, but their placement is different. It should be appreciated that the placement of these and other elements are only examples—they can be placed in many other positions on embodiments of medication adherence device.

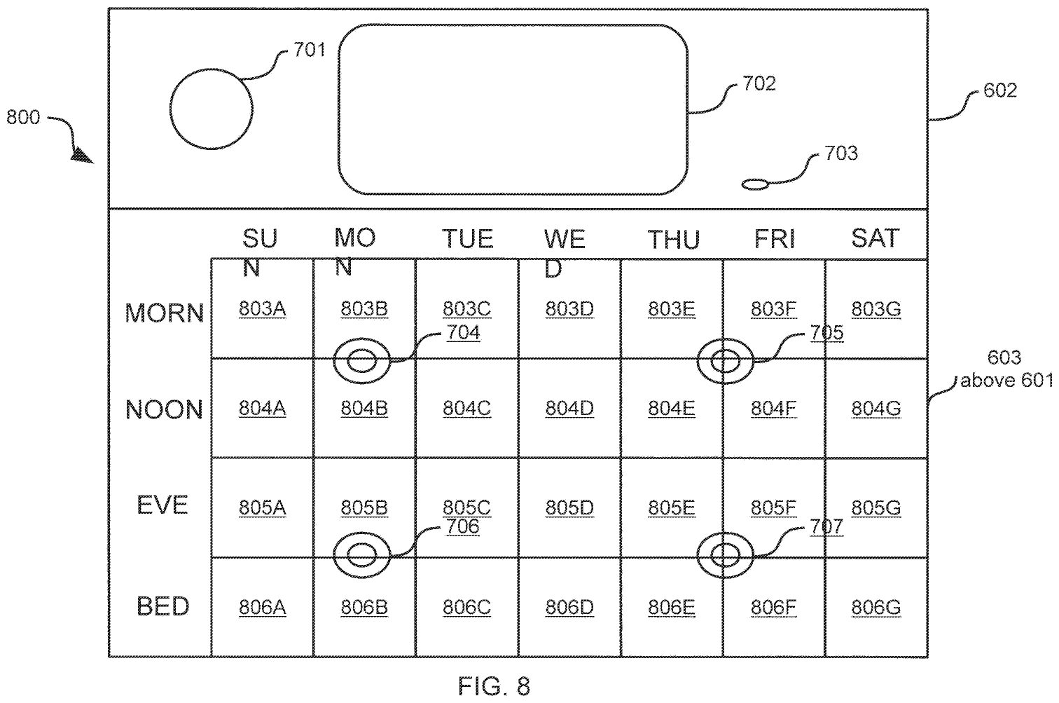

FIG. 8 is a simplified schematic drawing of medication unit 603 docked with the other components of the medication adherence device. Medication unit 603 now sits on top of camera unit 601 such that the cameras have a view of the medication compartments 803A thru 803G, 804A thru 804G, 805A thru 805G and 806A thru 806G.

Medication adherence devices, such as examples 300, 400, 500, 600, 700 and 800 shown in FIGS. 3-8, typically include familiar software and hardware components. For example, they may include operating systems, processors, local memory for storage, I/O devices, and system buses interconnecting the hardware components. RAM and disk drives are examples of local memory for storage of data and computer programs. Other types of local memory include magnetic storage media, optical storage media, flash memory, networked storage devices, and the like.

Networking capabilities are also included in the medication adherence devices, such as examples 300, 400, 500, 600, 700 and 800 shown in FIGS. 3-8. These capabilities may be wired, wireless, or a combination of wired and wireless technologies, including wi-fi and cellular/mobile networking. Networking capabilities allow the medication adherence devices to be remotely managed and integrated into a patient medication adherence system. Medication adherence devices may have also have near field communications (NFC) capabilities, allowing communications with other devices in their vicinity. For example, the medication adherence device may use NFC to determine the contents of a medication bottle in its vicinity that has an NFC readable label. As another example, the medication adherence device may have wireless connectivity (e.g., BLE) to biometric devices and can collect patient biometric readings from these devices, which may be analyzed by the medication adherence device or medication adherence service.

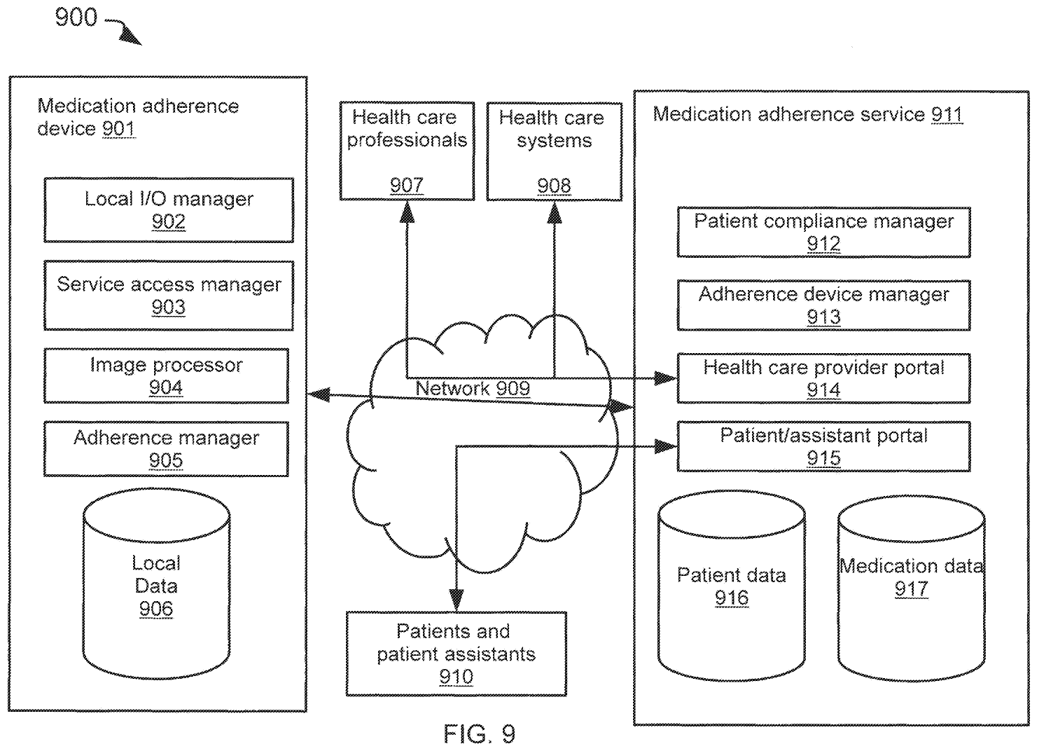

FIG. 9 illustrates an example of a medication adherence device 901 deployed in a system 900 for managing and monitoring patient medication usage. System 900 includes a medication adherence service 911. Medication adherence service 911 is a cloud-based software service, which receives information about patient medication prescriptions. In conjunction with medication adherence device 901, deployed for a patient, it manages and monitors patient medication usage. While only one medication adherence device 901 is shown in FIG. 9, medication adherence service 911 supports the monitoring of many patients and their associated medication adherence devices 901. FIG. 9 illustrates communication occurring over a network—this can be the public network, a private network, or a combination.

Examples of components that may be a part of the medication adherence service 911 are illustrated in FIG. 9. Two databases are represented: patient data 916 includes information about patients being managed by medication adherence service 911, including, for example, their identity, contact information, current prescriptions, identity of their physician(s) or other health care professionals managing their care, and any family members or other personal caregivers authorized to assist the patient. The identity of the medication adherence device 901, including the software and hardware version, currently in use by the patient is also included. Medication data 917 includes information about medications that may be prescribed to patients, including instructions for usage such as dosage and timing, as well as warnings doctors or pharmacists request the system to remind the patient of. Medication data 917 also may include images of medications, and other identifying information, including, for example, the color, shape, and size of medications. Medication data 917 may be received directly from health care providers or from other sources such as pharmacies. While two databases are illustrated, it will be appreciated that the information may be organized in a variety of manners.

Continuing with example modules that may be part of medication adherence service 911, health care provider portal 914 provides a secure interface for authorized health care professionals 907 and health care systems 908 to provide patient and patient medication information, enabling the system to manage and monitor patient medication adherence. Such information may be stored as patient data 916, as well as medication data 917. Health care provider portal 914 may also provide a web or application interface for authorized health care professionals 907 and systems 908 to monitor or manage patient medication adherence. For example, a health care professional, such as a nurse or doctor, may use the interface to view reports on the adherence of patients that are being managed on behalf of the health care professional. The interface may also be used, for example, to request an alert when a patient is not complying with their medication schedule. The nature of the alerts may be configurable, based upon factors such as the frequency, or the potential health impact, of non-adherence. Furthermore, the application interface of health care provider portal 914 supports machine-to-machine interfaces such as, for example, an EMR (Electronic Medical Record) system or a pharmacy portal, from which information can be sent or retrieved.

Patient/assistant portal 915 provides the patient and authorized assistants 910 an interface to view information about the patient's adherence, provide additional information or request alerts. Authorized assistants may, for example, include a spouse, an adult child, another relative, or a trusted friend or neighbor. Viewing adherence information or receiving alerts can make an authorized assistant aware of when the patient needs assistance.

Adherence device manager 913 interfaces over network 909 with medication adherence devices 901. It receives device identity and version information from such devices. The device manager receives messages from medication adherence devices and, where appropriate, routes them to other medication adherence service components, such as patient adherence manager 912. The device manager may also send outgoing communications from other medication adherence service components to medication adherence devices.

Patient adherence manager 912 uses patient data 916 and/or medication data 917 to create and execute patient medication adherence management and monitoring methods. This manager operates in conjunction with a medication adherence device 901 assigned to the patient. Patient adherence manager 912 may send data to the medication adherence device 901 that is specific to the particular patient the device is managing and monitoring.

We turn now to example components illustrated for medication adherence device 901. Local data 906 is local data that may be used by the device for managing and monitoring the patient's medication adherence. This includes patient specific information that is received from medication adherence service 911. For example, any given patient is prescribed a limited set of medications, which is only a small subset of all medications that might be prescribed to all of the patients managed and monitored by medication adherence service 911. Some or all of the information needed to manage and monitor patient medication adherence may be stored locally to reduce network traffic and allow the device to operate even when the medication adherence service is not reachable. Local data 906 may include the patient's medication schedule, as well as prompts or information related to the patient's medications that is to be provided to the patient. Local data 906 may also include medication information, such as an image of a medication or its shape, size, and color, which may be used by the medication adherence device in evaluating whether the patient is in adherence.

Adherence manager 905 evaluates the medication schedule of the user and schedules activities to manage and monitor the patient's medication adherence. The adherence manager evaluates patient adherence with the aid of image processor 904. Additionally, the adherence manager uses the local I/O manager 902 to interface with the patient using the medication adherence device.

Image processor 904 performs image processing to evaluate images collected by medication adherence device 901's cameras. For example, image processor 904 evaluates medication compartments to determine whether they are empty or contain medications. Further, the image processor may evaluate images to determine the quantity of specific medications in a compartment by comparing images of the medications with images of the specific medications that have been prescribed to the patient. These comparisons may also involve attributes such as the size, shape, and color of medications.

Service access manager 903 handles the connection of medication adherence device 901 to the medication adherence service. It is responsive to commands received from the medication adherence service 911 to perform operations, such as reporting the device's status and upgrading device software.

Local I/O manager 902 controls interfaces on the medication adherence device 901 such as any displays, speakers, LEDs, microphones, and cameras. The local I/O manager 902 may be directed by other components utilizing these interfaces. For example, the adherence manager 905 may direct the local I/O manager to play a prompt on the speaker. In another case, the adherence manager may turn on the microphone and collect audio input from the patient using medication adherence device 901.

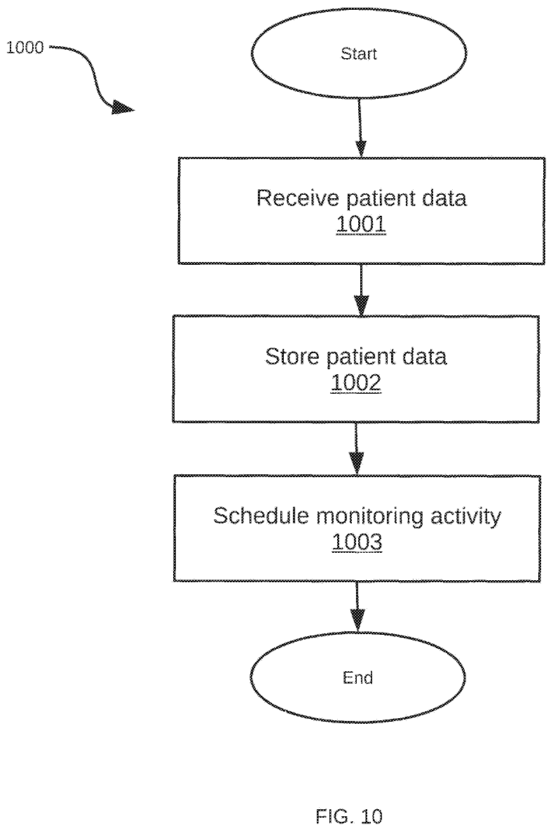

FIG. 10 illustrates an example of a method 1000 for provisioning a medication adherence system with a patient's medication adherence data. At 1001 patient data is received by, for example, the medication adherence service 911 in FIG. 9. Patient data may be received from a health care professional or from health care systems that are directed to send it to the medication adherence service. At 1002 the patient data is stored in the medication adherence service's databases. At 1003 the data is analyzed in terms of the patient's medication schedule and the system then begins to manage and monitor the patient's medication adherence. Patient specific information, such as a patient's medication schedule, may be sent from the medication adherence service to a medication adherence device 901 assigned to the patient so that the medication adherence device can then manage and monitor the patient medication adherence.

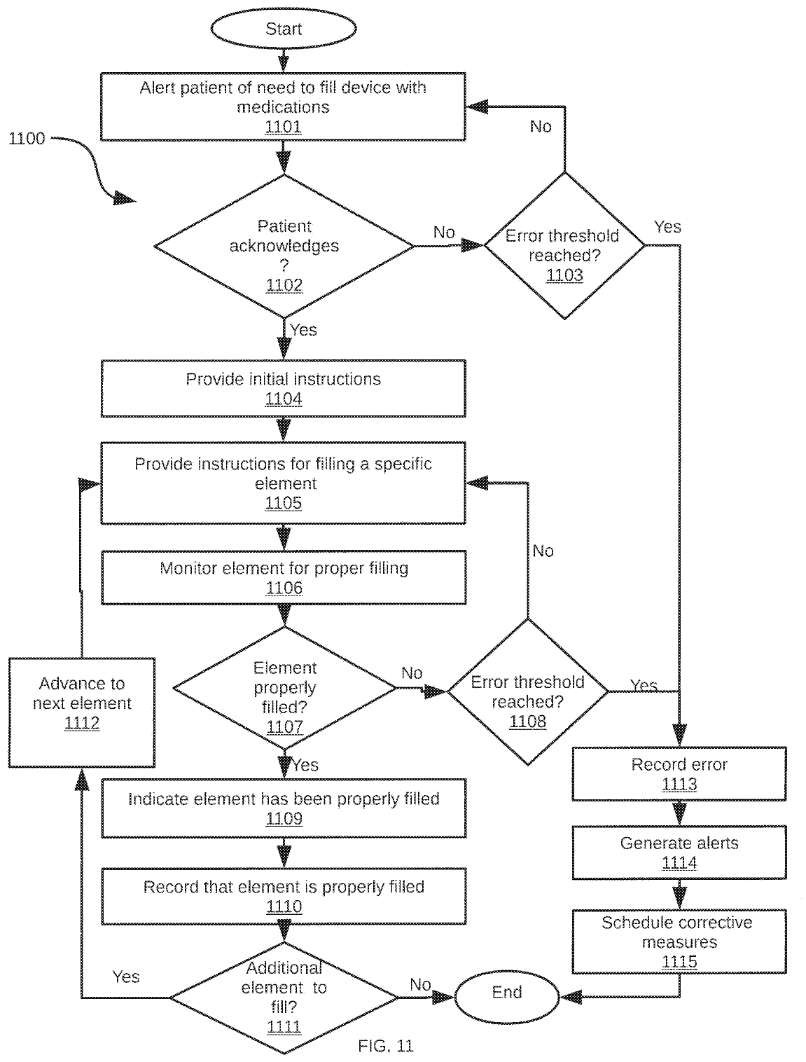

FIG. 11 illustrates an example method 1100 of filling a medication adherence device, such as the examples 300, 400, 500, 600, 700, 800 and 901 illustrated earlier, with medications. Starting at 1101 the patient is alerted of the need to fill the device with medications. Alternatively, the patient may initiate refilling of the medication adherence device prior to receiving the alert of 1101. For example, the patient may recognize that the medication unit is empty at the end of the week and use an interface, such as a button, to initiate refilling. In the case of patient initiation, steps 1101 and 1102 are unnecessary and may be forgone. When needed, the device may alert the patient by, for example, making an audio prompt, displaying a message or video on the device's display, or by lighting LEDs on the device. At 1102 the medication adherence device determines whether the patient has acknowledged the need to fill the device. The device waits for the patient to acknowledge the alert of 1101 before proceeding. For example, the acknowledgement may be received in audio form using the device's microphone, by touch screen input, by a button, or by other mechanisms. If, after some period of waiting, an acknowledgement has not been received at 1103 the device determines whether an error threshold has been reached. If not, the method returns to 1101 and repeats the alert asking the patient to fill the medications. If the error threshold has been reached the method continues at 1113, which will be outlined shortly.

Continuing with the case where the patient acknowledges the need to fill the device with medications, at 1104 the device provides initial instructions to the patient, such as an overview of the process of filling the device. At 1105 instructions are provided for filling a specific element of the device. An element may be a specific medication compartment, a particular row of compartments, or any other set of compartments that the device is configured to direct the patient to fill. This is advantageous because patients may prefer to fill their devices in a variety of manners. For example, one patient may prefer to fill each medication compartment completely, with all the medications that are to be placed in the compartment, before filling the next compartment. Another patient may prefer to fill an entire row of compartments with a single medication, especially when they take a medication at the same time each day. The medication adherence device can be configured to operate and provide instructions in the manner the patient, their assistant, or the health care professional managing their care prefers.

Having provided instructions, the medication adherence device monitors the element for proper filling. Using one or more cameras, the adherence device collects images of the element. The images may be analyzed to determine the number of medications in each of the one or more medication compartments comprising the element, as well as the size, shape, and color of these medications. This information can then be compared against specifications or images that the medication adherence device has stored locally, or it may be sent to a medication adherence service (for example, 911 in FIG. 9) for analysis. At 1107 a determination is made as to whether the element has been properly filled. If not, a determination is made at 1108 if error thresholds have been reached. If the error thresholds have not been reached, the method moves back to 1105 and provides further instructions. The earlier instructions may be repeated, or they may be altered, based on an assessment of progress to this point in completion of filling the element, to better aid the patient in filling the element. If the error threshold has been reached the method continues at 1113, which will be outlined shortly.

Continuing with the affirmative case at 1107, where the element has been properly filled, the method moves to 1109. An indication is given to the patient that the element has been properly filled using, for example, the device's speaker, display, or LEDs. At 1110 the device records the time of the element being properly filled. This information is sent to medication adherence service 911, so that the patient's records can be updated and available for reporting.

At 1111 the device examines the patient's medication schedule and the current state of the device's medication compartments. If there are additional elements that need to be filled, the method advances to 1112 to advance to the next such element. As noted earlier, an element may be a compartment, row of compartments or any other set of compartments. If, at 1111, there are no further elements the method ends.

Returning to 1113, which is executed following an error threshold being reached, the specific error is recorded with a timestamp. The error is reported to the medication adherence service 911. At 1114 any desired alerts are generated based upon the patient's configuration. Alerts may be sent to health care professionals or the patient's assistants. The delivery mechanism may vary depending upon preferences and the nature of the error—phone alerts, emails or text messages are examples of possible delivery mechanisms. At 1115 corrective measures are scheduled. The current method may be repeated immediately from the point of error, or at a scheduled time, in an attempt to properly refill the medication adherence device. In other cases, retry may be delayed until requested by a health care professional or patient assistant. This may have the benefit of minimizing patient frustration by delaying a repeat of the method until someone has consulted with the patient regarding how to correct the error.

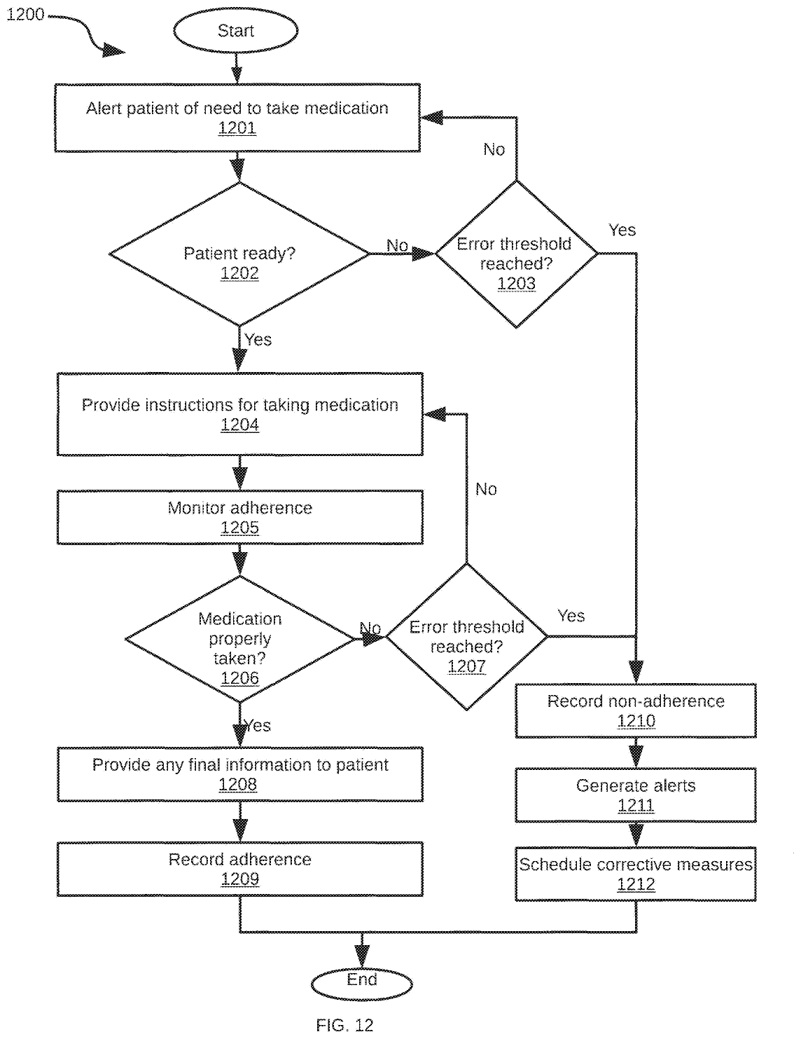

FIG. 12 illustrates an example method 1200 of alerting and monitoring a patient regarding medication adherence, using a medication adherence device such as those in the examples 300, 400, 500, 600, 700 and 901. The method is executed when the patient is scheduled to take a medication. At 1201 the patient is alerted of the need to take a medication by, for example, making an audio prompt, displaying a message or video on the device's display, illuminating the device or one or more of its medication compartments, or by lighting LEDs on the device. At 1202 the device waits for the patient to acknowledge the alert of 1201 before proceeding. For example, the device may be received in audio form using the device's microphone, by touch screen input, by a button, or by other mechanism. An explicit acknowledgement of alert 1201 may not be required. For example, a patient may provide an implicit acknowledgement by emptying a medication compartment. If, after some period of waiting, an acknowledgement has not been received at 1203 the device determines whether an error threshold has been reached. If not, the method returns to 1201 and repeats the alert reminding the patient that they need to take their medication. If the error threshold has been reached the method continues at 1210, which will be outlined shortly.

Continuing with the case where the patient is ready, at 1204 instructions are given to the patient. These may be provided in audio form, using the device's display, or LEDs on the device or a combination. LEDs may, for example, be lit for the compartment that the patient should take medications from in conjunction with audio instructions. Instructions may be configured and customized for the patient, as requested by, for example, the health care professional managing their care. Customized instructions can help the patient overcome difficulties they may be having in properly taking their medications.

At 1205 the medication adherence device monitors the patient taking medications. Using one or more cameras, the adherence device collects images of the device's medication compartments. At 1206 the images are analyzed to determine whether the patient is properly taking the medication by examining whether they have removed all of the medications from the proper compartment. Examples of not properly taking the medications include: 1) no medications being removed from the compartment; 2) only a portion of the medications being removed from the proper compartment; and 3) medications being removed from the wrong compartment. If the medications have not been properly taken, the method proceeds to 1207 to determine whether an error threshold has been reached. Some errors, such as removing medications from the wrong compartment, may be at threshold immediately because of their potential consequences. Other errors, such as only removing some or none of the medications from the proper compartment, may warrant retry. If a threshold has not been reached for the particular error the method moves back to 1201 and provides further instructions. The earlier instructions may be repeated, or they may be altered, based on the error, to better aid the patient in properly taking their medication. If the error threshold has been reached the method continues at 1210, which will be outlined shortly.

Continuing with the case where medication has been properly taken, the method proceeds to 1208 where any final instructions or information are given to the patient. They may be based upon requests from the health care professional administering the patient's care and the medication being taken. As an example, some medications may require that the patient not eat anything for a period of time after taking the medication; therefore, an audio or display message may be given to the patient to remind them of this. At 1209 the device records the time of the medication being properly taken. This information is sent to medication adherence service 911, so that the patient's records can be updated and available for reporting.

Returning to 1210, which is executed following an error threshold being reached, the specific error is recorded with a timestamp. The error is reported to the medication adherence service 911 and the patient's records are updated to indicate that they are not adhering to their medication schedule. At 1211 any desired alerts are generated based upon the patient's configuration. Alerts may be sent to health care professionals or the patient's assistants. The delivery mechanism may vary depending upon preferences and the nature of the error—phone alerts, emails or text messages are examples of possible delivery mechanisms. At 1212 corrective measures are scheduled. The current method may be repeated immediately from the point of error, or at a scheduled time, in an attempt to keep the patient on their medication schedule. In some cases, the patient's prescription may indicate that if too much time has passed since the medication was prescribed to be taken it should simply be skipped; the method schedules any retry based upon this. In some cases, retry may be delayed until requested by a health care professional or patient assistant. This may have the benefit of delaying a repeat of the method until someone has consulted with the patient regarding how to correct the error.

It should be appreciated that the specific steps illustrated in FIGS. 10-12 provide particular methods according to some embodiments. Other sequences of steps may also be performed according to alternative embodiments. For example, alternative embodiments may perform the steps outlined above in a different order. Moreover, the individual steps illustrated in FIGS. 10-12 may include multiple sub-steps that may be performed in various sequences. Furthermore, additional steps may be added or removed depending on the particular application.

It should be appreciated that some embodiments may be implemented by hardware, software, firmware, middleware, microcode, hardware description languages, or any combination thereof. When implemented in software, firmware, middleware, or microcode, the program code or code segments to perform the necessary tasks may be stored in a computer-readable medium such as a storage medium. Processors may be adapted to perform the necessary tasks. The term “computer-readable medium” includes, but is not limited to, portable or fixed storage devices, optical storage devices, wireless channels, sim cards, other smart cards, and various other non-transitory mediums capable of storing, containing, or carrying instructions or data.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means “including but not limited to”. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

What is claimed is:

1. A medication adherence device, comprising:
a camera unit comprising a camera;
a medication unit configured to be docked and undocked with the camera unit, wherein the medication unit comprises a plurality of medication compartments for organizing medications corresponding to tablets according to a prescribed medication schedule, wherein the medication unit comprises one or more light emitting diodes (LEDs) associated with the respective medication compartments and the medication compartments are within a field of view of the camera; and
an interface unit configured to control the one or more LEDs based on the prescribed medication schedule.

2. The medication adherence device of claim 1, wherein the interface unit includes a wireless interface configured to wirelessly control the one or more LEDs in response to the medication unit being undocked from the camera unit.

3. The medication adherence device of claim 1, wherein the camera unit is positioned above the medication unit.

4. The medication adherence device of claim 1, wherein the interface unit comprises a speaker and/or a display configured to provide alerts based on the prescribed medication schedule.

5. The medication adherence device of claim 1, wherein the interface unit comprises:
an additional camera unit configured to identify an object within a field of view outside of the medication unit; and
a speaker and/or a display configured to provide alerts based on the identified object.

6. The medication adherence device of claim 1, wherein the camera is configured to capture one or more images of the medication compartments in response to docking the medication unit with the camera unit.

7. The medication adherence device of claim 1, wherein the interface unit includes a local I/O manager configured to control the one or more LEDs based on contents of the respective medication compartments.

8. The medication adherence device of claim 1, wherein the interface unit is configured to control the one or more LEDs based on an error associated with adherence to the prescribed medication schedule.

9. The medication adherence device of claim 1, wherein the camera unit comprises a number of cameras and wherein the number of cameras is based on a distance between the respective cameras of the camera unit and a bottom of the medication unit.

10. The medication adherence device of claim 1, wherein the camera unit comprises a number of cameras and wherein the number of cameras is based on a viewing angle between the respective cameras of the camera unit with respect to a bottom of the medication unit.

11. A medication adherence system, comprising:
a camera unit comprising one or more cameras;
a medication unit configured to be docked and undocked with the camera unit, wherein the medication unit comprises a plurality of medication compartments for organizing medications corresponding to tablets in respective medication compartments according to a prescribed medication schedule, wherein the medication unit comprises a number of light emitting diodes (LEDs) associated with the respective medication compartments, and wherein the one or more cameras are configured to capture one or more images of the medication compartments and the medication compartments are within a field of view of the one or more cameras; and
an interface unit configured to control each of the one or more LEDs based on the prescribed medication schedule and the one or more images of the medication compartments captured by the camera unit.

12. The medication adherence system of claim 11, wherein the interface unit includes a wireless interface configured to wirelessly control the one or more LEDs in response to the medication unit being undocked from the camera unit.

13. The medication adherence system of claim 11, wherein the camera unit is positioned above the medication unit.

14. The medication adherence system of claim 11, wherein the interface unit comprises a speaker and/or a display configured to provide alerts based on the prescribed medication schedule.

15. The medication adherence system of claim 11, wherein the interface unit comprises:
an additional camera unit configured to identify an object within a field of view outside of the medication unit; and
a speaker and/or a display configured to provide alerts based on the identified object.

16. The medication adherence system of claim 11, wherein the one or more cameras are configured to capture the one or more images of the medication compartments in response to docking the medication unit with the camera unit.

17. The medication adherence system of claim 11, wherein the interface unit includes a local I/O manager configured to control the one or more LEDs based on a medication level associated with the medication compartments.

18. The medication adherence system of claim 11, wherein the interface unit is configured to control the one or more LEDs based on an error associated with adherence to the prescribed medication schedule.

19. The medication adherence system of claim 11, wherein the camera unit comprises a number of cameras and wherein the number of cameras is based on a distance between the respective cameras of the camera unit and a bottom of the medication unit.

20. The medication adherence system of claim 11, wherein the camera unit comprises a number of cameras and wherein the number of cameras is based on a viewing angle between the respective cameras of the camera unit with respect to a bottom of the medication unit.
